(19) United States
(12) Patent Application Publication
(10) Pub. No.: US 2025/0040825 A1
(43) Pub. Date: Feb. 6, 2025

(54) MEASURING DEVICE FOR THE NON-INVASIVE DETERMINATION OF A BLOOD GLUCOSE CONCENTRATION IN THE BODY OF A MAMMAL

(71) Applicant: UNIVERSITAT DUISBURG-ESSEN, Essen (DE)

(72) Inventors: Mandana JALALI, Dusseldorf (DE); Andreas PROKSCHA, Duisburg (DE); Tobias KUBICZEK, Marl (DE); Jan Taro SVEJDA, Dusseldorf (DE); Sascha PREU, Muhltal (DE); Thomas KAISER, Duisburg (DE); Jan BALZER, Mulheim an der Ruhr (DE); Daniel ERNI, Duisburg (DE)

(73) Assignee: UNIVERSITAT DUISBURG-ESSEN, Essen (DE)

(21) Appl. No.: 18/792,627

(22) Filed: Aug. 2, 2024

(30) Foreign Application Priority Data
Aug. 3, 2023 (DE) 10 2023 207 437.0

(51) Int. Cl. A61B 5/0507; A61B 5/00; A61B 5/145

(52) U.S. Cl. CPC A61B 5/0507; A61B 5/14532; A61B 5/6826; A61B 2560/0462

(57) ABSTRACT

The invention relates to a measuring device for the non-invasive determination of a blood sugar concentration in the body of a mammal, comprising a housing (G), a measuring electronics (IC) in the housing (G), wherein the measuring electronics (IC) further comprises at least one transmitter (Tx) for emitting high-frequency signals with a frequency of more than 0.1 THz and less than 10 THz, at least one receiver (Rx) for receiving high-frequency signals with a frequency of more than 0.1 THz and less than 10 THz, wherein the at least one transmitter (Tx) is designed such that during operation it emits the high-frequency signal via an antenna (ANT_Tx) integrated in the measuring device, wherein the at least one receiver (Rx) is designed such that, during operation, it receives a high-frequency signal via an antenna (ANT_Rx) integrated in the measuring device, wherein the measuring device is configured to be attached relative to a nail plate (NP) on a nail of a finger or toe of the mammal, wherein the high-frequency signals from at least one transmitter (Tx) are coupled through the nail plate (NP) into the nail bed (NB) of the mammal during operation, and wherein the high-frequency signals backscattered from the nail bed (NB) of the mammal through the nail plate (NP) during operation are received by at least one receiver (Rx), wherein the measuring device further comprises an evaluation device (CPU) which is configured to evaluate the backscattered high-frequency signals received by at least one receiver (Rx) in order to determine the blood sugar concentration in the body of a mammal.

The present application claims priority to German Patent Application No. 10 2023 207 437.0, filed Aug. 3, 2023, which is incorporated in its entirety by reference herein.

The invention relates to a measuring device for the non-invasive determination of a blood sugar concentration in the body of a mammal.

### BACKGROUND OF THE INVENTION

For people suffering from diabetes as well as for competitive athletes, it is important to know their current blood sugar level. Diabetes is an enormous problem, since it is now assumed that one in ten adults worldwide suffers from diabetes.

Blood sugar is frequently measured invasively by taking a drop of blood from a blood vessel. For this purpose the skin and an underlying venous blood vessel is usually punctured with a lancet, whereupon blood flows out. The drop of blood is then placed on a test element for further analysis. These methods include the amperometric measurement method.

However, such invasive procedures are disadvantageous in several respects. On the one hand, even with good hygiene, there is always the risk that viruses or bacteria will penetrate the skin and/or the blood vessel system at the puncture site, on the other hand, repeated puncturing also causes damage to the vessel, so that after a series of punctures at the same location, the vessel can no longer be punctured. In addition, invasive extraction also poses a psychological problem. In general, quality of life suffers.

Against this background, approaches have already been sought in the past that allow a non-invasive determination of blood sugar concentration in the body of a mammal.

Exemplary methods that have been described theoretically for this purpose are methods based on dielectric spectroscopy.

As examples reference is made to the articles “Pendra goes Dutch: lessons for the CE mark in Europe” by the authors I. M. E. Wentholt, J. B. L. Hoekstra, A. Zwart and J. H. DeVries published in Diabetologia (2005) 48: 1055-1058, DOI 10.1007/s00125-005-1754-y, “First human experiments with a novel non-invasive, non-optical continuous glucose monitoring system” by the authors A. Caduff, E. Hirt, Yu. Feldman, Z. Ali, and L. Heinemann, published in Biosensors and Bioelectronics 19 (2003) 209/217, and “Non-invasive glucose monitoring in patients with diabetes: A novel system based on impedance spectroscopy” by the authors A. Caduff, F. Dewarrat, M. Talary, G. Stalder, L. Heinemann and Yu. Feldman, published in Biosensors and Bioelectronics 22 (2006) 598-604, which describe impedance spectroscopy in the frequency range 1-200 MHz at the skin surface.

Also known as examples are approaches using special fringing-field contact electrodes and multi-scale numerical skin models as mentioned in the article “Sensitivity and specificity analysis of fringing-field dielectric spectroscopy applied to a multi-layer system modelling the human skin” by the authors Sonja Huclova, Dirk Baumann, Mark S Talary and Jürg Fröhlich, published in Phys. Med. Biol. 56 (2011) 7777-7793 doi:10.1088/0031-9155/56/24/007 or “PENDRA: The Once and Future Noninvasive Continuous Glucose Monitoring Device?” by the author STUART ALAN WEINZIMER, published in DIABETES TECHNOLOGY & THERAPEUTICS, Volume 6, Number 4, 2004, pages 442-444.

These approaches have proven difficult to implement since the environmental variations (humidity, sweat, . . . ) in the dielectric function of the outermost layer of the skin (stratum corneum) are too great for a precise estimation of the blood glucose level.

Other approaches, such as GHz transmission measurements, allow non-invasive glucose measurement using transmission and reflection measurements at frequencies around 30 GHz. This makes it possible in principle to calibrate out (de-embedding) the highly variable outermost layers of skin. A sensor clip for transmission measurements through the earlobe or the thumb-index finger skin fold has already been developed, see the article “Compact Non-Invasive Millimeter-Wave Glucose Sensor” by the authors Peter H. Siegel, Adrian Tang, Gabriel Virbila, Yanghyo Kim, M. C. Frank Chang and Victor Pikov, published in 2015 40th International Conference on Infrared, Millimeter, and Terahertz waves (IRMMW-THz), Hong Kong, China, 2015, pp. 1-3, doi: 10.1109/IRMMW-THz.2015.7327413. and is also the subject of a U.S. Pat. No. 11,229,383B2.

Other alternative approaches involve THz transmission measurements, in which the insertion loss is measured in a prism-coupled manner by means of THz-TDS along the skin surface as a function of the glucose content, see for example the article “Noninvasive blood glucose monitoring in the terahertz frequency range” by the authors Olga Cherkasova, Maxim Nazarov, and Alexander Shkurinov, published in Opt Quant Electron (2016) 48:217 DOI 10.1007/s11082-016-0490-5.

Another optical approach is multi-spectral sensing by means of a wearable arrangement consisting of several IR to multi-colour LED sources with a photodetector placed next to them on the skin surface for multispectral transmission measurements, as is known from the article “Wearable-band type visible-near infrared optical biosensor for non-invasive blood glucose monitoring” by the authors Vega Pradana Rachim and Wan-Young Chung, published in Sensors & Actuators: B. Chemical 286 (2019) 173-180.

Also known is the photothermal measurement of glucose content, in which light very specifically heats the glucose molecules in the skin, followed by temperature measurement. Such devices from DiaMon Tech AG, Berlin, with various form factors (tabletop device, pocket sensor, arm-wrist sensor) are currently in the testing phase.

The problem with all approaches is the strong variation in the electrical/optical behaviour of the outermost layer of skin. This has a negative impact not only on the outermost layer of skin itself, but also on the interface between a sensor and the outermost layer of skin, e.g. due to sweat, moisture, etc.

For approaches based on a transmission measurement in the GHz range, a force-fitting measuring clamp is also required.

Other methods could in principle provide improvement, but require considerable compensation and/or computational effort.

Starting from this situation, it is the object of the invention to provide a measuring device for the non-invasive determination of a blood sugar concentration in the body of a mammal, which makes it possible to precisely non-invasively measure a blood sugar concentration in the body of a mammal.

The object is achieved by a device according to Claim 1. Further advantageous embodiments are in particular the subject of the dependent claims.

The invention is explained in more detail below with reference to the figures. In these figures:

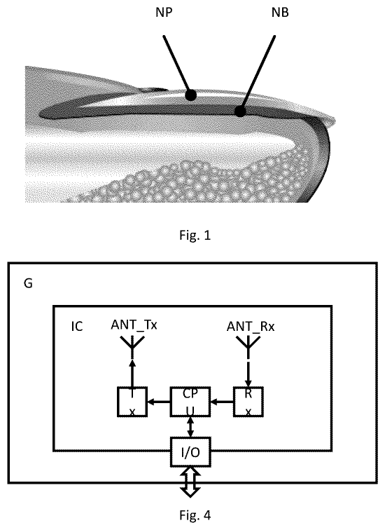

FIG. 1 shows a basic structure of a human finger with nail bed and nail plate,

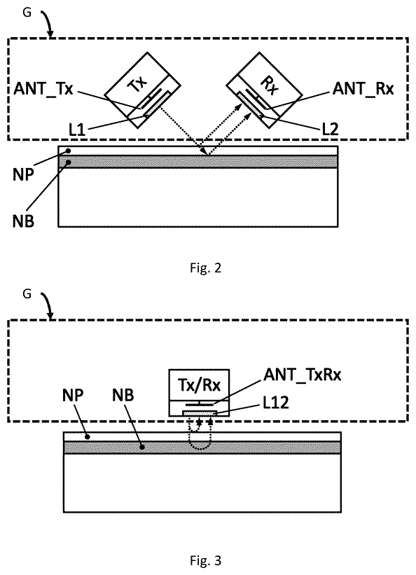

FIG. 2 shows an exemplary schematic bistatic embodiment of the invention,

FIG. 3 shows an exemplary schematic monostatic embodiment of the invention, and

FIG. 4 shows exemplary schematic functional units of a measuring device according to the invention.

The invention will be described in more detail below with reference to the figures. It should be noted that different aspects are described, each of which can be used individually or in combination. That is to say that any aspect can be used with different embodiments of the invention unless explicitly shown as a pure alternative.

Furthermore, for the sake of simplicity, reference will generally only be made to one entity. Unless explicitly noted, the invention can also comprise several of the relevant entities. In this respect, the use of the words “a”, “an” and “one” is to be understood only as an indication that in a simple embodiment at least one entity is used.

Where procedures are described hereinafter, the individual steps of a procedure can be arranged and/or combined in any order, unless explicitly indicated otherwise by the context. Furthermore, the procedures can be combined with one another, unless expressly indicated otherwise.

Information with numerical values is generally not to be understood as exact values but also includes
a tolerance of +/−1% up to +/−10%.

References to standards or specifications are to be understood as references to standards or specifications that are/were in force at the time of the application and/or—if priority is claimed—at the time of the priority application. However, this is not to be understood herewith as a general exclusion of applicability to subsequent or replacing standards or specifications.

FIG. 4 shows schematically functional units of a measuring device according to the invention for non-invasively determining a blood sugar concentration in the body of a mammal. Mammal is to be understood here in particular as a human, but use on animals in an analogous manner is not excluded.

The measuring device according to the invention has a housing G and a measuring electronics IC, which can consist, for example, of one or more integrated circuits or one or more photonic integrated circuits or a combination of both, in the housing G.

The measuring electronics IC further comprises a transmitter Tx for emitting high-frequency signals with a frequency of more than 0.1 THz and less than 10 THz and a receiver Rx for receiving high-frequency signals with a frequency of more than 0.1 THz and less than 10 THz, wherein the transmitter Tx is designed such that, during operation, it emits the high-frequency signal via an antenna ANT_Tx integrated in the measuring device on-chip or in the housing G, and wherein the receiver Rx is designed such that, during operation, it receives a high-frequency signal via an antenna ANT_Rx integrated in the measuring device on-chip or in the housing G.

The measuring device, i.e., the housing G with the measuring electronics IC, is configured to be attached to a finger or toe of the mammal relative to a nail plate NP. An attachment can be provided by adhesion (e.g., with a plaster), clamping (e.g., a fastening device arranged on the housing G), wrapping (e.g. bandage, elastic band), etc.

When the measuring device is in operation, the high-frequency signals from the transmitter Tx are coupled through the nail plate NP into the nail bed NB of the mammal by means of the integrated antenna ANT_Tx. The high-frequency signals backscattered from the nail bed NB of the mammal through the nail plate NP during operation are received by the receiver Rx by means of the integrated antenna ANT_Rx.

Furthermore, the measuring device has an evaluation device CPU, which is configured to evaluate the backscattered high-frequency signals received by the receiver Rx in order to determine the blood sugar concentration in the body of a mammal.

Furthermore, the measuring device has an interface to an external device, e.g. a computer or a mobile phone or another display device (e.g. display), which can display the measured values.

By means of the measuring device according to the invention it is possible to make a non-invasive blood sugar determination by means of high-frequency reflection measurements and in particular THz reflection measurements of the perfused nail bed through the nail plate NP of the nail (e.g. toenail or fingernail). The method is based on the glucose-dependent reflection measurement method and allows the glucose concentrations or their changes to be determined in the underlying, well-perfused nail bed NB.

The invention takes advantage of the fact that, unlike the skin on the ear/earlobe or a skin fold, the nail bed is generally particularly well perfused, whilst no sweat forms at the interfaces and is also generally dimensionally stable, so that the classic problems from the prior art can be avoided. In addition, the invention allows for an extremely compact and stable structure.

In particular, the nail bed NB has a significantly simpler tissue layer structure than the skin (growth zone and nail bed matrix), which has a much stronger and denser blood supply, since the nail bed is rich in blood and lymph vessels. Thus, the likelihood of finding blood—and therefore glucose—is significantly higher.

In addition, the nail bed NB is very smooth and well defined at the interface with the nail plate NP and—unlike the skin—has no pores, wrinkles, hairs or sweat glands. Any modifications to the fingernail or toenail, such as nail polish or artificial fingernails, are usually made of terahertz-transparent materials, so that this does not or only slightly restricts measurability.

Furthermore, the nail bed NB is naturally protected against environmental influences by the fingernail plate NP. Thus, the variations in the nail bed NB at the interface with the nail plate NP are significantly reduced compared to variations on the skin surface.

In addition, by means of reflectometry a simpler structure can be provided compared to transmission/reflection measurement.

The use of high frequencies and especially THz frequencies is advantageous since the nail bed NB, viewed through the fingernail plate NP, has an absorption maximum in the frequency range of 0.2-0.4 THz.

At these frequencies, simple integrated antennae ANT_Tx or ANT_Rx can also be integrated together with the transmitter Tx and the receiver Rx on the measuring electronics IC, so that particularly compact measuring devices of different sizes can be provided. The arrangement in a measuring electronics IC also allows embodiments that are permanently in a damp/wet environment, so that, for example, a swimmer/diver can also continuously measure his blood sugar level. Unlike with optical sensors, no optical window is required. The measuring device according to the invention can therefore be carried and used at all times and anywhere.

The invention makes it possible, in particular, to provide very broadband and/or multi-frequency measurements to increase sensitivity. Other parameters such as inflammatory markers (leukocyte density, interleukin, etc.) can also be determined based on the same measuring principle. Further details on this can be found in the (not yet published) article “Non-invasive, easy to implement glucose sensing via the fingernail bed using THz radiation” by the authors Mandana Jalali, Andreas Prokscha, Yijun Yun, Tobias Kubiczek, Thomas Kaiser, Jan Balzer, and Daniel Erni in Biomedical Engineering/Biomedizinische Technik, 2023.

Using known/measured dielectric functions (permittivity, conductivity) of the layers of a fingernail, a material model can be constructed using a multipolar Cole-Cole model for both the nail plate NP and the nail bed NB.

In one embodiment of the invention, a first focusing or matching element L1 (FIG. 2) or L12 (FIG. 3) is arranged downstream of the antenna of the transmitter ANT_Tx, which is irradiated before high-frequency signals penetrate into the nail bed NB during operation.

In a further embodiment of the invention, a second focussing or matching element L2 (FIG. 2) or L12 (FIG. 3) is arranged upstream of the antenna of the receiver ANT_Rx, which during operation is irradiated by the signals backscattered from the nail bed NB.

According to one embodiment of the invention, the focussing or matching element L1 and/or the focussing or matching element L2 or L12 (FIG. 3) comprises an environmentally resistant plastic, in particular polymethyl pentene, or a material with low absorption of the frequency range used in operation, in particular highly resistive silicon.

According to another embodiment of the invention—see FIG. 3—the transmitter Tx and the receiver Rx are realized as a component Tx/Rx in order to emit and receive signals of more than 0.1 THz and less than 10 THz.

In yet another embodiment of the invention, the antenna ANT_Tx integrated in the measuring device for emitting the high-frequency signal is also simultaneously configured as an antenna ANT_Rx integrated in the measuring device for receiving. In FIG. 3, this common antenna is designated as ANT_TxRx.

In yet another embodiment of the invention, the focussing or matching element L1 integrated in the measuring device for emitting the high-frequency signal is also at the same time configured as a focussing or matching element L2 for receiving. In FIG. 3, this common element is designated as L12.

According to a further embodiment of the invention, the underlying measuring principle in monostatic operation is THz reflectometry (FIG. 3).

According to a further embodiment of the invention, the underlying measuring principle in bistatic operation is THz ellipsometry at one or more angles (FIG. 2).

In yet another embodiment of the invention, the data evaluation of the signals from the receiver Rx is carried out using an electromagnetic model of the layered fingernail structure and/or by means of AI-based pattern recognition.

In yet another embodiment of the invention, the measuring device is integrated into an artificial fingernail that can be applied to the fingernail.

Without limiting the generality, the measuring device can provide an integrated evaluation and control CPU. Under the control of the CPU—for example a microprocessor or microcontroller—the duration and the time interval of successive measurements can be determined (measurement profile), e.g. depending on one or more previously measured values. The CPU can be integrated in the housing G or located on a separate device that communicates with the measuring device via an interface, e.g. a communication unit I/O (e.g. near field communication (NF), Bluetooth, WLAN, USB connection or similar). The CPU can also have databases and data evaluation programs or can access them.

Likewise, the measuring device can have an energy collection unit which collects energy through movement of the measuring device in order to enable operation.

Furthermore, the measuring device can have a communication unit I/O, with which measured values can be made available or measurement profiles can be programmed. The communication unit I/O can be either wired or wireless. The communication unit I/O can be configured either as a separate component in the housing G or as part of the measuring electronics IC.

1. A measuring device for the non-invasive determination of a blood sugar concentration in the body of a mammal, comprising
a housing,
a measuring electronics in the housing, wherein the measuring electronics further comprises:
at least one transmitter for emitting high-frequency signals with a frequency of more than 0.1 THz and less than 10 THz,
at least one receiver for receiving high frequency signals with a frequency of more than 0.1 THz and less than 10 THz,
wherein the at least one transmitter is configured such that, during operation, it emits the high-frequency signal via an antenna integrated in the measuring device,
wherein the at least one receiver is configured such that, during operation, it receives a high-frequency signal via an antenna integrated in the measuring device,
wherein the measuring device is configured to be attached relative to a nail plate on a nail of a finger or toe of the mammal,
wherein the high-frequency signals from at least one transmitter are coupled through the nail plate into the nail bed of the mammal during operation, and wherein the high-frequency signals backscattered from the nail bed of the mammal through the nail plate during operation are received by the receiver,
wherein the measuring device further comprises an evaluation device which is configured to evaluate the backscattered high-frequency signals received by at least one receiver in order to determine the blood sugar concentration in the body of a mammal.

2. The measuring device according to claim 1, wherein a first focussing or matching element is arranged downstream of the antenna of the at least one transmitter, which is irradiated before high-frequency signals penetrate into the nail bed during operation.

3. The measuring device according to claim 1, wherein a second focussing or matching element is arranged upstream of the antenna of the at least one receiver, which, during operation, is irradiated by signals backscattered from the nail bed.

4. The measuring device according to claim 2, wherein the focussing or matching element comprises an environmentally resistant plastic.

5. The measuring device according to claim 1, wherein the at least one transmitter and the at least one receiver are realized as one component in order to emit and receive signals of more than 0.1 THz and less than 10 THz.

6. The measuring device according to claim 1, wherein the antenna integrated in the measuring device for emitting the high-frequency signal is at the same time also configured as an antenna integrated in the measuring device for receiving.

7. The measuring device according to claim 1, wherein the focussing or matching element integrated in the measuring device for emitting the high-frequency signal is at the same time also configured as a focussing or matching element for receiving.

8. The measuring device according to claim 1, wherein the underlying measuring principle in monostatic operation is THz reflectometry.

9. The measuring device according to claim 1, wherein the underlying measuring principle in bistatic operation is THz ellipsometry.

10. The measuring device according to claim 1, wherein the data evaluation of the signals from at least one receiver is carried out using an electromagnetic model of the layered fingernail structure and/or by means of AI-based pattern recognition.

11. The measuring device according to claim 1, wherein the measuring device is integrated into an artificial fingernail that is capable of being applied to the fingernail.

12. The measuring device according to claim 2, wherein the focussing or matching element comprises polymethyl pentene.

13. The measuring device according to claim 2, wherein the focussing or matching element comprises a material with low absorption in the frequency range used in operation.

14. The measuring device according to claim 2, wherein the focussing or matching element comprises a highly resistive silicon.

15. The measuring device according to claim 2, wherein a second focussing or matching element is arranged upstream of the antenna of the at least one receiver, which, during operation, is irradiated by signals backscattered from the nail bed, whereby the focussing or matching element comprises an environmentally resistant plastic, whereby the at least one transmitter and the at least one receiver are realized as one component in order to emit and receive signals of more than 0.1 THz and less than 10 THz, whereby the antenna integrated in the measuring device for emitting the high-frequency signal is at the same time also configured as an antenna integrated in the measuring device for receiving, whereby the focussing or matching element integrated in the measuring device for emitting the high-frequency signal is at the same time also configured as a focussing or matching element for receiving, whereby the underlying measuring principle in monostatic operation is THz reflectometry and in bistatic operation is THz ellipsometry, whereby the data evaluation of the signals from at least one receiver is carried out using an electromagnetic model of the layered fingernail structure and/or by means of AI-based pattern recognition, and whereby the measuring device is integrated into an artificial fingernail that is capable of being applied to the fingernail.

16. The measuring device according to claim 15, wherein the focussing or matching element comprises polymethyl pentene.

17. The measuring device according to claim 15, wherein the focussing or matching element comprises a material with low absorption in the frequency range used in operation.

18. The measuring device according to claim 15, wherein the focussing or matching element comprises a highly resistive silicon.
